(19) United States
(12) Patent Application Publication
(10) Pub. No.: US 2002/0119531 A1
(43) Pub. Date: Aug. 29, 2002

(54) Prostate-associated protease antibody

(76) Inventors: Olga Bandman, Mountain View, CA (US); Preeti G. Lal, Santa Clara, CA (US)

Correspondence Address: INCYTE GENOMICS, INC., LEGAL DEPARTMENT, 3160 PORTER DRIVE, PALO ALTO, CA, 94304, US

(21) Appl. No.: 09/988,975

(22) Filed: Nov. 19, 2001

Related U.S. Application Data
(63) Continuation-in-part of application No. 09/478,957, filed on Jan. 7, 2000, now Pat. No. 6,350,448.
(63) Continuation-in-part of application No. 08/807,151, filed on Feb. 27, 1997, now Pat. No. 6,043,033.

(51) Int. Cl. C07K 16/40; C12P 21/04; C12N 9/00

(52) U.S. Cl. 435/70.21; 530/388.26; 435/183

(57) ABSTRACT

The present invention provides a human prostate-associated protease (HUPAP), polynucleotides which encode HUPAP and antibodies which specifically bind HUPAP. The invention also provides expression vectors, host cells, agonists, antagonists, and antisense molecules. The invention also provides methods for producing and using HUPAP and for treating disorders associated with expression of HUPAP.

This application is a continuation-in-part of U.S. Ser. No. 09/478,957, filed Jan. 7, 2000, and U.S. Ser. No. 08/807,151, filed Feb. 27, 1997, which issued as U.S. Pat. No. 6,043,033, on Mar. 28, 2000.

### FIELD OF THE INVENTION

This invention relates to a human prostate-associated protease, its encoding polynucleotide, and antibodies which specifically bind the protein and to the use of these molecules in the diagnosis, prognosis, treatment and evaluation of therapies for disorders of the prostate and gastrointestinal system.

### BACKGROUND OF THE INVENTION

Prostate-specific antigen (PSA) is a 33 kD glycoprotein synthesized in the epithelial cells of the prostate gland. It is a secreted serine protease of the kallikrein family. PSA has been shown to digest the seminal vesicle protein, semenogelin, parathyroid hormone-related protein, and insulin-like growth factor-binding protein-3 (Henttu et al. (1994) Ann Med 26:157-164; Cramer et al. (1996) J Urol 156:526-531).

Genes encoding the three human kallikreins, tissue kallikrein (KLK1), glandular kallikrein (KLK2), and PSA are located in a cluster at chromosome map position 19q13.2-q13.4 (Riegmen (1992) Genomics 14:6-11). PSA shares more extensive homology with KLK2 than with KLK1. Both PSA and KLK2 are produced by prostate epithelial cells, and their expression is regulated by androgens. Three amino acid residues were found to be critical for serine protease activity, residues H₆₅, D₁₂₀, and S₂₁₃ in PSA (Bridon et al. (1995) Urology 45:801-806). Substrate specificity, described as chymotrypsinogen-like (with KLK2) or trypsin-like (with PSA) is thought to be determined by S₂₀₇ in PSA and D₂₀₉ in KLK2 (Bridon, supra). KLK1 is chymotrypsinogen-like and expressed in the pancreas, urinary system, and sublingual gland. KLK1, like the other kallikreins, is made as a pre-pro-protein and is processed into an active form of 238 amino acids by cleavage of a 24 amino acid terminal signal sequence (Fukushima et al. (1985) Biochemistry 24:8037-8043).

Adenocarcinoma of the prostate accounts for a significant number of malignancies in men over 50, and over 122,000 new cases occur per year in the United States alone. Prostate-specific antigen (PSA) is the most sensitive marker available for monitoring cancer progression and response to therapy. Serum PSA is elevated in up to 92% of patients with prostatic carcinoma, and serum concentration depends upon tumor volume. Since PSA is also moderately elevated in patients with benign prostate hyperplasia, additional techniques are needed to distinguish between the two.

The enterokinases (also called enteropeptidases) are a functionally distinct family of serine proteases with homology to PSA and the kallikreins. Enterokinases act in a multi-step, enzymatic cascade that allows the digestion of exogenous macromolecules without destroying similar endogenous material. This cascade results in the conversion of pancreatic proenzymes to active enzymes in the lumen of the gut. Trypsin, chymotrypsin, and carboxypeptidase A are examples of pancreatic enzymes activated by intestinal enterokinases. Enterokinase has a high specificity for the amino acid sequence (Asp)₄-Lys, a motif found in the amino-termini of trypsinogens from a wide range of species. Congenital deficiency in enterokinase may cause life-threatening intestinal malabsorption.

The catalytic subunit of bovine enterokinase was cloned and characterized by LaVallie et al. (1993; J Biol Chem 268:23311-23317). The bovine enterokinase is a serine protease with four predicted intramolecular disulfide bonds, sharing homology with other serine proteases, such as the kallikreins and hepsin. Like the kallikreins, bovine enterokinase has characteristic active site histidine, aspartic acid, and serine residues at conserved positions.

Discovery of a novel protein related to PSA, bovine enterokinase, human pancreatic kallikrein, and rat renal kallikrein; its encoding polynucleotide; and antibodies which specifically bind the protein satisfies a need in the art by providing molecules which are useful in the diagnosis, prognosis, treatment and evaluation of therapies for disorders of the prostate and gastrointestinal system.

### SUMMARY OF THE INVENTION

The present invention features a novel human prostate-associated kallikrein, hereinafter designated HUPAP, its encoding polynucleotide, and antibodies which specifically bind the protein which are useful in the diagnosis, prognosis, treatment and evaluation of therapies for disorders of the prostate and gastrointestinal system.

The invention provides a purified HUPAP having the amino acid sequence shown in SEQ ID NO:1. The invention also provides isolated polynucleotides that encode HUPAP and the complements of these polynucleotides. One of these polynucleotides has the nucleic acid sequence of SEQ ID NO:2 and the complement thereof. The invention further provides expression vectors and host cells comprising polynucleotides that encode HUPAP.

The invention provides a method for producing HUPAP using a host cell, and methods for using the protein. In one aspect, the invention provides a method for treating prostate disorders including prostate cancer and benignprostatic hyperplasia by administering an antibody or an antagonist to HUPAP. In another aspect, the invention provides a method for treating gastrointestinal disorders such as congenital enterokinase deficiency by administering HUPAP or an agonist to HUPAP. In addition, the invention features methods for treating cancers of the esophagus, stomach, small intestine, large intestine and colon; pancreatitis; and ulcerative colitis by administering an antibody or antagonist to HUPAP. The present invention also provides compositions comprising HUPAP or antibodies, agonists, and antagonists which specifically bind to HUPAP which may be used therapeutically. The invention further provides an array containing HUPAP.

The invention provides a method for using a protein to screen a plurality of antibodies to identify an antibody which specifically binds the protein comprising contacting a plurality of antibodies with the protein under conditions to form an antibody:protein complex, and dissociating the antibody from the antibody:protein complex, thereby obtaining antibody which specifically binds the protein.

The invention also provides methods for using a protein to prepare and purify polyclonal and monoclonal antibodies which specifically bind the protein. The method for preparing a polyclonal antibody comprises immunizing a animal with protein under conditions to elicit an antibody response, isolating animal antibodies, attaching the protein to a substrate, contacting the substrate with isolated antibodies under conditions to allow specific binding to the protein, dissociating the antibodies from the protein, thereby obtaining purified polyclonal antibodies. The method for preparing a monoclonal antibodies comprises immunizing a animal with a protein under conditions to elicit an antibody response, isolating antibody producing cells from the animal, fusing the antibody producing cells with immortalized cells in culture to form monoclonal antibody producing hybridoma cells, culturing the hybridoma cells, and isolating monoclonal antibodies from culture.

The invention further provides purified antibodies which bind specifically to a protein. The invention also provides a method for using an antibody to detect expression of a protein in a sample, the method comprising combining the antibody with a sample under conditions for formation of antibody:protein complexes; and detecting complex formation, wherein complex formation indicates expression of the protein in the sample. In one aspect, the amount of complex formation when compared to standards is diagnostic of a disorder of the prostate or gastrointestinal system.

The invention still further provides a method for immunopurification of a protein comprising attaching an antibody to a substrate, exposing the antibody to a sample containing protein under conditions to allow antibody:protein complexes to form, dissociating the protein from the complex, and collecting purified protein. The invention yet still further provides an array containing an antibody which specifically binds HUPAP.

### BRIEF DESCRIPTION OF THE FIGURES

FIGS. 1A and 1B show the amino acid sequence (SEQ ID NO:1) and the encoding polynucleotide sequence (SEQ ID NO:2) of HUPAP. The alignment was produced using MACDNASIS PRO software (Hitachi Software Engineering, San Bruno Calif.).

FIG. 2 shows the amino acid sequence alignments among HUPAP (SEQ ID NO:1), bovine enterokinase (g416132; SEQ ID NO:6), human pancreatic kallikrein (g186653; SEQ ID NO:7), and African rat renal kallikrein (g55527; SEQ ID NO:8). The alignment was produced using the MEGALIGN program of LASERGENE software (DNASTAR, Madison Wis.).

FIG. 3 shows the hydrophobicity plot (MACDNASIS PRO software) for HUPAP, SEQ ID NO: 1; the positive X axis reflects amino acid position, and the negative Y axis, hydrophobicity.

FIG. 4 shows the hydrophobicity plot for human pancreatic kallikrein, SEQ ID NO:7.

### DESCRIPTION OF THE INVENTION

Before the present proteins, polynucleotides, and methods are described, it is understood that this invention is not limited to the particular methodology, protocols, cell lines, vectors, and reagents described as these may vary. It is also to be understood that the terminology used herein is for the purpose of describing particular embodiments only and is not intended to limit the scope of the present invention which will be limited only by the appended claims.

It must be noted that as used herein and in the appended claims, the singular forms “a”, “an”, and “the” include plural reference unless the context clearly dictates otherwise. For example, reference to “a host cell” includes a plurality of such host cells, reference to the “antibody” is a reference to one or more antibodies known to those skilled in the art.

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods, devices, and materials are now described. All publications mentioned herein are incorporated herein by reference for the purpose of describing and disclosing the cell lines, vectors, and methodologies which are reported in the publications which might be used in connection with the invention. Nothing herein is to be construed as an admission that the invention is not entitled to antedate such disclosure by virtue of prior invention.

### DEFINITIONS

“Agonist” refers to a molecule or compound which, when bound to HUPAP, causes a change which modulates the activity of HUPAP. Agonists may include proteins, nucleic acids, carbohydrates, or any other molecules or compounds which bind to HUPAP and increase its lifespan or activity.

An “allele” is an alternative form of a gene which may result from at least one mutation in the polynucleotide.

“Antagonist” or “inhibitor” refers to a molecule or compound which, when bound to HUPAP, blocks or modulates the biological or immunological activity of HUPAP. Antagonists and inhibitors may include proteins, nucleic acids, carbohydrates, or any other molecules or compounds which bind to HUPAP and decrease its lifespan or activity..

“Antibody” refers to intact immunoglobulin molecule, a polyclonal antibody, a monoclonal antibody, a chimeric antibody, a recombinant antibody, a humanized antibody, single chain antibodies, a Fab fragment, an F(ab)₂ fragment, an Fv fragment; and an antibody-peptide fusion protein.

“Antigenic determinant” refers to an immunogenic epitope, structural feature, or region of an oligopeptide, peptide, or protein which is capable of inducing formation of an antibody which specifically binds the protein.

“Array” refers to an ordered arrangement of at least two polynucleotides, proteins, or antibodies on a substrate. At least one of the polynucleotides, proteins, or antibodies represents a control or standard, and the other polynucleotide, protein, or antibody of diagnostic or therapeutic interest. The arrangement of two to about 40,000 polynucleotides, proteins, or antibodies on the substrate assures that the size and signal intensity of each labeled complex, formed between each polynucleotide and at least one nucleic acid, each protein and at least one ligand or antibody, or each antibody and at least one protein to which the antibody specifically binds, is individually distinguishable. “Biologically active” refers to a protein having structural, regulatory, or biochemical functions of a naturally occurring molecule.

The “complement” of a polynucleotide refers to a nucleic acid sequence which is completely complementary to the polynucleotide over its full length and which will hybridize to an mRNA under conditions of high stringency.

A “composition” refers to the polynucleotide and a labeling moiety; a purified protein and a pharmaceutical carrier or a heterologous, labeling or purification moiety; an antibody and a labeling moiety; and the like.

“Consensus” refers to a polynucleotide which has been extended using XL-PCR kit (Applied Biosystems (ABI), Foster City Calif.) in the 5′ and/or the 3′ direction and resequenced, which has been assembled from the overlapping nucleic acid sequences from more than one Incyte clone using the GELVIEW fragment assembly system (GCG, Madison Wis.), or which has been both extended and assembled.

“Derivative” refers to a polynucleotide or a protein that has been subjected to a chemical modification. Derivatization of a polynucleotide can involve substitution of a nontraditional base such as queosine or of an analog such as hypoxanthine. Derivatization of a protein involves the replacement of a hydrogen by an acetyl, acyl, alkyl, amino, formyl, or morpholino group. Derivative molecules retain the biological activities of the naturally occurring molecules but may confer advantages such as longer lifespan or enhanced activity.

“Disorders” refers to conditions, diseases, and disorders including benign prostatic hyperplasia, congenital enterokinase deficiency, pancreatitis, ulcerative colitis and cancers, particularly adenocarcinomas and sarcomas, of the prostate, pancreas, and gastrointestinal system (esophagus, stomach, small intestine, large intestine, and colon).

“Fragment” refers to a chain of consecutive nucleotides from about 50 to about 4000 base pairs in length or to a portion of an antibody which specifically binds the protein. Nucleotide fragments may be used in amplification or hybridization technologies to identify related nucleic acid molecules and in binding assays to screen for a ligand. Such ligands are useful as therapeutics to regulate replication, transcription or translation. Antibody fragments are useful in detection and in purification of the protein having the amino acid sequence of SEQ ID NO:1.

“Humanized antibody” refers to antibodies in which amino acids have been replaced in the non-antigen binding regions so that the molecule more closely resembles a human antibody while still retaining the original binding ability.

“Identity” as applied to amino acid or nucleic acid sequences, refers to the quantification (usually percentage) of nucleotide or residue matches between at least two sequences aligned using a standardized algorithm such as Smith-Waterman alignment (Smith and Waterman (1981) J Mol Biol 147:195-197), CLUSTALW (Thompson et al. (1994) Nucleic Acids Res 22:46734680), or BLAST2 (Altschul et al (1997) Nucleic Acids Res 25:3389-3402). BLAST2 may be used in a standardized and reproducible way to insert gaps in one of the sequences in order to optimize alignment and to achieve a more meaningful comparison between them. “Similarity” as applied to proteins uses the same algorithms but takes into account conservative substitutions of nucleotides or residues and produces a higher percent value than identity.

“Immunologically active” refers to the capability of the natural, recombinant, or synthetic HUPAP, or any oligopeptide thereof, to induce antibody formation as part of a specific immune response in cells or animals. Biological activity is not a prerequisite for immunogenicity.

“Labeling moiety” refers to any reporter molecule or visible or radioactive label than can be attached to or incorporated into a polynucleotide, protein, or antibody. Visible labels include but are not limited to anthocyanins, green fluorescent protein (GFP), β glucuronidase, luciferase, Cy3 and Cy5, and the like. Radioactive markers include radioactive forms of hydrogen, iodine, phosphorous, sulfur, and the like.

“Modulation” refers to any change, increase or decrease, in the biological, functional, binding, or immunological properties or activities of HUPAP.

“Oligonucleotides” or “oligomers” refer to a nucleic acid sequence of at least about 10 nucleotides and as many as about 60 nucleotides which can be used in PCR technologies.

“Peptide nucleic acid” refers to a molecule which comprises an oligonucleotide to which an amino acid residue, such as lysine, and an amino group have been added.

“Polynucleotide” refers to a cDNA, nucleic acid molecule, nucleotide sequence, or fragments thereof, that may be single- or double-stranded DNA or RNA, sense or antisense, of genomic or synthetic origin.

“Portion” refers to a fragment of a protein which may range in size from four amino acid residues to the entire amino acid sequence minus one residue.

Reporter molecules may include radionuclides, enzymes, fluorescent, chemiluminescent, or chromogenic agents as well as substrates, cofactors, inhibitors, magnetic particles, and the like.

“Protein” refers to an amino acid sequence, peptide, or polypeptide, and portions thereof, that are naturally occurring, recombinantly produced, or synthetic. “HUPAP” refers to a purified protein obtained from any species, particularly mammalian including bovine, equine, murine, ovine, porcine, and preferably human, from any source whether natural, synthetic, semi-synthetic, or recombinant.

“Specific binding” refers to a special and precise interaction between two molecules which is dependent upon their structure, particularly their molecular side groups. For example, the intercalation of a regulatory protein into the major groove of a DNA molecule or the binding between an epitope of a protein and an agonist, antagonist, or antibody.

“Substrate” refers to any rigid or semi-rigid support to which polynucleotides, proteins, or antibodies are bound and includes magnetic or nonmagnetic beads; capillaries; chips; fibers; filters; gels; membranes; microparticles with a variety of surface forms including wells, trenches, pins, channels and pores; phages; plates; polymers; slides; glass, metal, paper, plastic rubber or other tubing; and wafers.

“Variant” refers to molecules that are recognized variations of a polynucleotide or a protein encoded by the polynucleotide. Splice variants may be determined by BLAST score, wherein the score is at least 100, and most preferably at least 400. Allelic variants have a high percent identity to the polynucleotides and may differ by about three bases per hundred bases. “Single nucleotide polymorphism” (SNP) refers to a change in a single base as a result of a substitution, insertion or deletion. The change may be conservative (purine for purine) or non-conservative (purine to pyrimidine) and may or may not result in a change in an encoded amino acid or its secondary, tertiary, or quaternary structure.

### The Invention

The invention is based on the discovery of a novel prostate-associated protease, its encoding polynucleotide, and antibodies which specifically bind the protein and to the use of these molecules in the diagnosis, prognosis, treatment and evaluation of therapies for disorders of the prostate and gastrointestinal system.

The polynucleotides encoding human HUPAP were first identified in Incyte clone 556016 from the spinal cord tissue cDNA library (SCORNOT01) through a computer-generated search for amino acid sequence alignments. A consensus sequence, SEQ ID NO:2, was derived from the following overlapping and/or extended nucleic acid sequences: Incyte clones 556016 (SCORNOT01), 842889 (PROSTUT05), and 991163 (COLNNOT11), which are SEQ ID NOs:3-5, respectively.

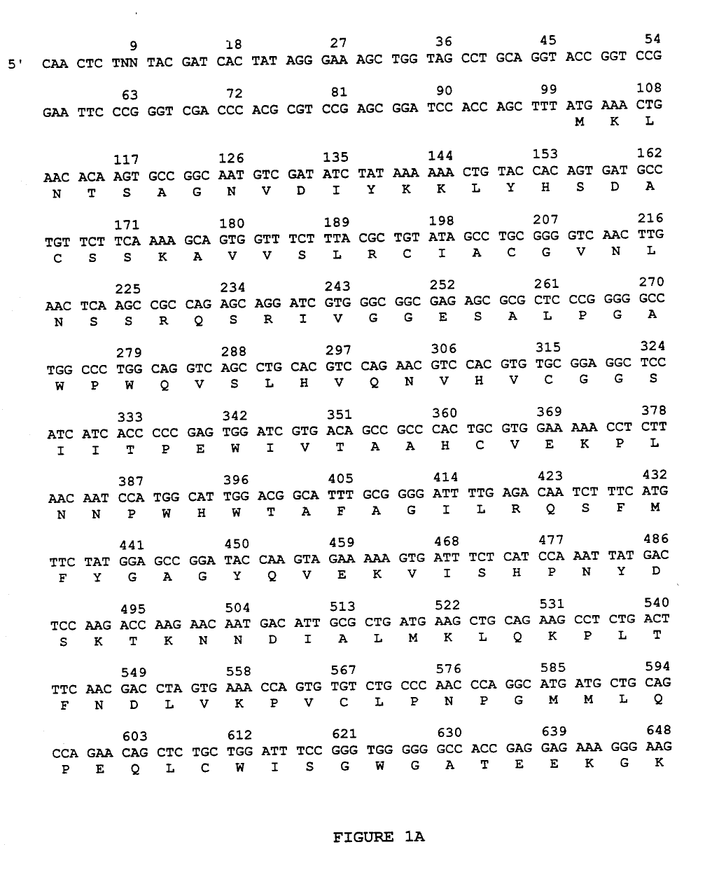

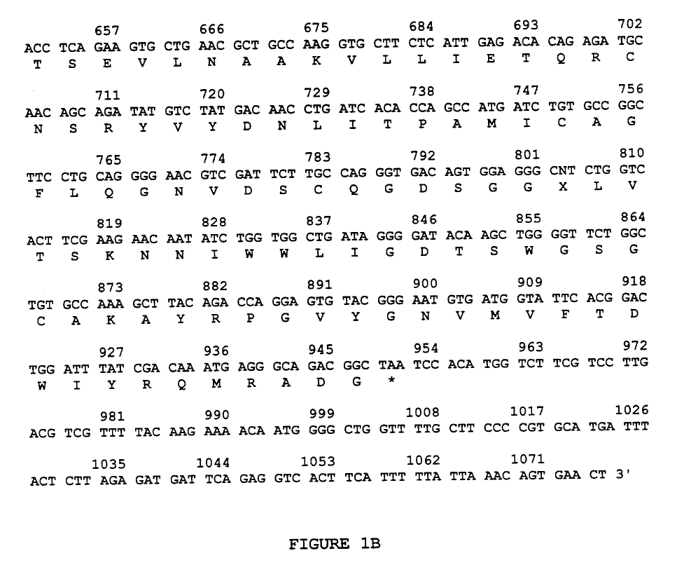

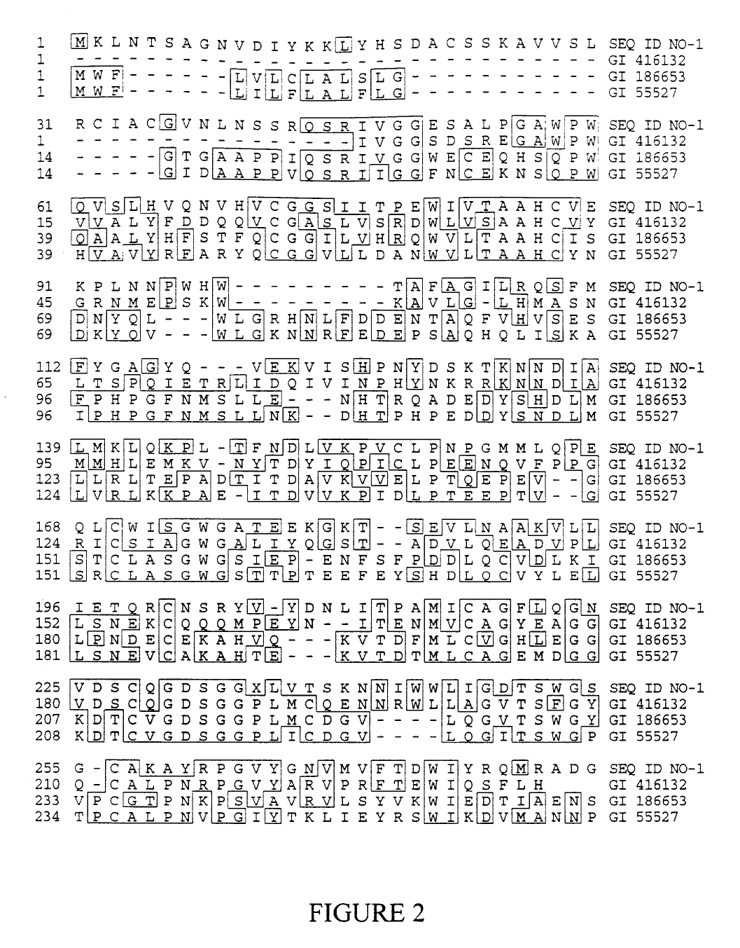

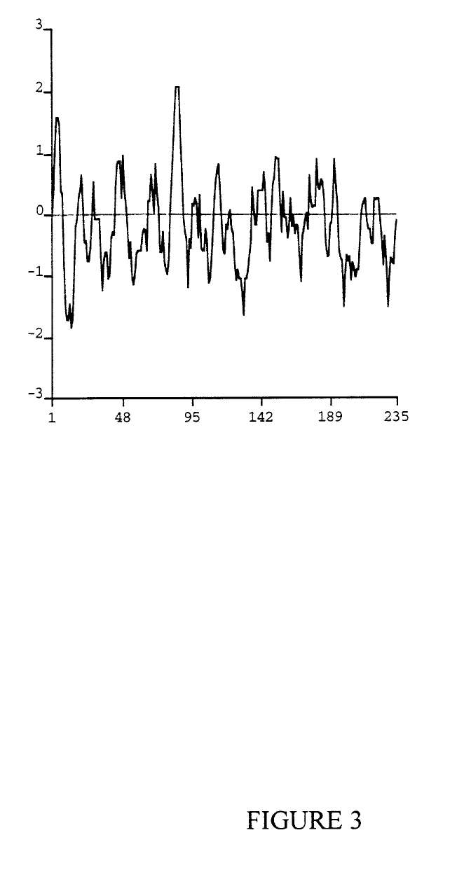

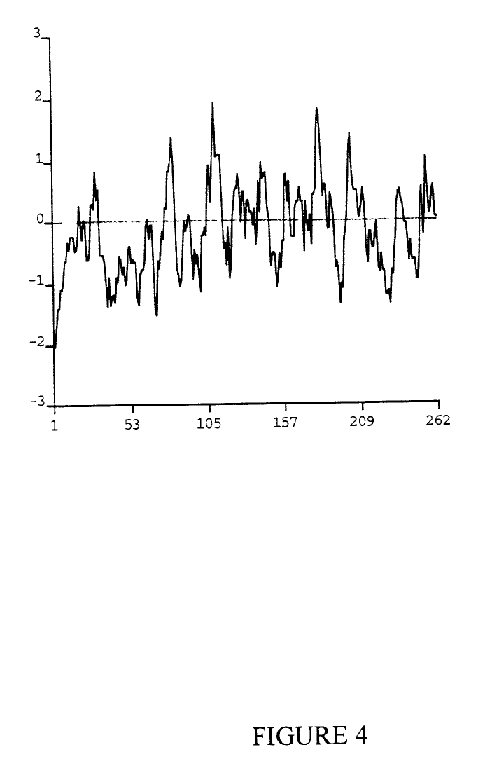

In one embodiment, the invention encompasses a protein comprising the amino acid sequence of SEQ ID NO:1. As shown in FIGS. 1A and 1B, HUPAP is 283 amino acids in length and has a potential N-glycosylation sites at asparagine residue 4. HUPAP has chemical and structural homology with bovine enterokinase (g416132; SEQ ID NO:6), human pancreatic kallikrein (gl86653; SEQ ID NO:7), and African rat renal kallikrein (g55527; SEQ ID NO:8). In particular, HUPAP and bovine enterokinase share 38% identity. The amino acid sequence from C₂₂ to S₄₅ near HUPAP's amino terminus is hydrophilic and resembles sequences important for membrane attachment or secretion (FIGS. 2, 3, and 4). As shown in FIG. 2, HUPAP also contains conserved residues, H₈₇, D₁₃₈, and S₂₃₂, which are critical for serine protease activity and amino acid residue D₂₂₆ which is likely to confer chymotrypsinogen-like activity on HUPAP. The HUPAP amino acid sequence includes eight conserved cysteine residues at positions 72, 88, 156, 170, 201, 217, 228, and 256 of SEQ ID NO:1. In the serine proteases mentioned above, these cysteines are structurally important and form four disulfide bonds. As illustrated by FIGS. 3 and 4, HUPAP and human pancreatic kallikrein have rather similar hydrophobicity plots. Northern analysis reveals expression of this sequence in the prostate, colon, and pancreas. Of seven tissue samples in which HUPAP was expressed, four were from prostate, and three of these four were from cancer patients.

The invention also encompasses variants of HUPAP having at least 80%, and more preferably 90%, and most preferably 95% amino acid sequence similarity to the amino acid sequence of SEQ ID NO:1.

### Characterization and Use of the Invention

### cDNA Libraries

In a particular embodiment disclosed herein, mRNA is isolated from cells and tissues using methods which are well known to those skilled in the art and used to prepare the cDNA libraries. The Incyte clones were isolated from cDNA libraries prepared as described in the EXAMPLES. The consensus sequences are chemically and/or electronically assembled from sequence fragments including Incyte cDNAs and extension and/or shotgun sequences using computer programs such as PHRAP (P Green, University of Washington, Seattle Wash.), and the AUTOASSEMBLER application (ABI). After verification of the 5′ and 3′ sequence, at least one of the representative cDNAs which encodes HUPAP is designated a reagent. In this case, the reagent cDNA is SEQ ID NO:5, Incyte clone 991163H1, from the colon cDNA library, COLNNOT11. Reagent cDNAs are also used in the construction of human microarrays.

### Sequencing

Methods for sequencing nucleic acids are well known in the art and may be used to practice any of the embodiments of the invention. These methods employ enzymes such as the Klenow fragment of DNA polymerase I, SEQUENASE, Taq DNA polymerase and thermostable T7 DNA polymerase (Amersham Pharmacia Biotech (APB), Piscataway N.J.), or combinations of polymerases and proofreading exonucleases such as those found in the ELONGASE amplification system (Life Technologies, Gaithersburg Md.). Preferably, sequence preparation is automated with machines such as the MICROLAB 2200 system (Hamilton, Reno Nev.) and the DNA ENGINE thermal cycler (MJ Research, Watertown Mass.). Machines commonly used for sequencing include the PRISM 3700, 377 or 373 DNA sequencing systems (ABI), the MEGABACE 1000 DNA sequencing system (APB), and the like. The sequences may be analyzed using a variety of algorithms well known in the art and described in Ausubel et al. (1997; Short Protocols in Molecular Biology, John Wiley & Sons, New York N.Y., unit 7.7) and Meyers (1995; Molecular Biology and Biotechnology, Wiley VCH, New York N.Y., pp. 856-853).

Shotgun sequencing may also be used to complete the sequence of a particular cloned insert of interest. Shotgun strategy involves randomly breaking the original insert into segments of various sizes and cloning these fragments into vectors. The fragments are sequenced and reassembled using overlapping ends until the entire sequence of the original insert is known. Shotgun sequencing methods are well known in the art and use thermostable DNA polymerases, heat4abile DNA polymerases, and primers chosen from representative regions flanking the sequence of interest. Incomplete assembled sequences are inspected for identity using various algorithms or programs such as CONSED (Gordon (1998) Genome Res 8:195-202) which are well known in the art. Contaminating sequences, including vector or chimeric sequences, or deleted sequences can be removed or restored, respectively, organizing the incomplete assembled sequences into finished sequences.

### Extension of a Nucleic Acid Sequence

The sequences of the invention may be extended using various PCR-based methods known in the art. For example, the XL-PCR kit (ABI), nested primers, and commercially available cDNA or genomic DNA libraries may be used to extend the nucleic acid sequence. For all PCR-based methods, primers may be designed using commercially available software to be about 22 to 30 nucleotides in length, to have a GC content of about 50% or more, and to anneal to a target molecule at temperatures from about 55 C. to about 68 C. When extending a sequence to recover regulatory elements, it is preferable to use genomic, rather than cDNA libraries.

### Hybridization

The polynucleotide and fragments thereof can be used in hybridization technologies for various purposes. A probe may be designed or derived from unique regions such as the 5′ regulatory region or from a nonconserved region (i.e., 5′ or 3′ of the nucleotides encoding the conserved catalytic domain of the protein) and used in protocols to identify naturally occurring molecules encoding HUPAP, allelic variants, or related molecules. The probe may be DNA or RNA, may be single-stranded, and should have at least 50% sequence identity to a nucleic acid sequence of SEQ ID NO:2. Hybridization probes may be produced using oligolabeling, nick translation, end-labeling, or PCR amplification in the presence of a reporter molecule. A vector containing the polynucleotide or a fragment thereof may be used to produce an mRNA probe in vitro by addition of an RNA polymerase and labeled nucleotides. These procedures may be conducted using commercially available kits.

The stringency of hybridization is determined by G+C content of the probe, salt concentration, and temperature. In particular, stringency can be increased by reducing the concentration of salt or raising the hybridization temperature. Hybridization can be performed at low stringency with buffers, such as 5×SSC with 1% sodium dodecyl sulfate (SDS) at 60 C., which permits the formation of a hybridization complex between nucleic acid sequences that contain some mismatches. Subsequent washes are performed at higher stringency with buffers such as 0.2×SSC with 0.1 % SDS at either 45 C. (medium stringency) or 68 C. (high stringency). At high stringency, hybridization complexes will remain stable only where the nucleic acids are completely complementary. In some membrane-based hybridizations, preferably 35% or most preferably 50%, formamide can be added to the hybridization solution to reduce the temperature at which hybridization is performed, and background signals can be reduced by the use of detergents such as Sarkosyl or TRITON X-100 (Sigma-Aldrich, St Louis Mo.) and a blocking agent such as denatured salmon sperm DNA. Selection of components and conditions for hybridization are well known to those skilled in the art and are reviewed in Ausubel (supra) and Sambrook et al. (1989) Molecular Cloning, A Laboratory Manual, Cold Spring Harbor Press, Plainview N.Y.

Arrays incorporating polynucleotides, proteins, or antibodies may be prepared and analyzed using methods well known in the art. Oligonucleotides or polynucleotides may be used as hybridization probes or targets to monitor the expression level of large numbers of genes simultaneously or to identify genetic variants, mutations, and single nucleotide polymorphisms. Proteins may be used to identify ligands, to investigate protein:protein interactions, or to produce a proteomic profile of gene expression (i.e., to detect and quantify expression of a protein in a sample). Antibodies may be also be used produce a proteomic profile of gene expression. Such arrays may be used to determine gene function; to understand the genetic basis of a condition, disease, or disorder; to diagnose a condition, disease, or disorder; and to develop and monitor the activities of therapeutic agents. (See, e.g., Brennan et al. (1995) U.S. Pat. No. 5,474,796; Schena et al. (1996) Proc Natl Acad Sci 93:10614-10619; Heller et al. (1997) Proc Natl Acad Sci 94:2150-2155; Heller et al. (1997) U.S. Pat. No. 5,605,662; and deWildt et al. (2000) Nature Biotechnol 18:989-994.)

Hybridization probes are also useful in mapping the naturally occurring genomic sequence. The probes may be hybridized to a particular chromosome, a specific region of a chromosome, or an artificial chromosome construction. Such constructions include human artificial chromosomes (HAC), yeast artificial chromosomes (YAC), bacterial artificial chromosomes (BAC), bacterial P1 constructions, or the cDNAs of libraries made from single chromosomes.

### Expression

Any one of a multitude of polynucleotides encoding the HUPAP may be cloned into a vector and used to express the protein, or portions thereof, in host cells. The polynucleotide can be engineered by such methods as DNA shuffling, as described in U.S. Pat. No. 5,830,721, and site-directed mutagenesis to create new restriction sites, alter glycosylation patterns, change codon preference to increase expression in a particular host, produce splice variants, extend half-life, and the like. The expression vector may contain transcriptional and translational control elements (promoters, enhancers, specific initiation signals, and polyadenylated 3′ sequence) from various sources which have been selected for their efficiency in a particular host. The vector, polynucleotide, and regulatory elements are combined using in vitro recombinant DNA techniques, synthetic techniques, and/or in vivo genetic recombination techniques well known in the art and described in Sambrook (supra, ch. 4, 8, 16 and 17).

A variety of host systems may be transformed with an expression vector. These include, but are not limited to, bacteria transformed with recombinant bacteriophage, plasmid, or cosmid DNA expression vectors; yeast transformed with yeast expression vectors; insect cell systems transformed with baculovirus expression vectors; plant cell systems transformed with expression vectors containing viral and/or bacterial elements, or animal cell systems (Ausubel supra, unit 16). For example, an adenovirus transcription/translation complex may be utilized in mammalian cells. After sequences are ligated into the E1 or E3 region of the viral genome, the infective virus is used to transform and express the protein in host cells. The Rous sarcoma virus enhancer or SV40 or EBV-based vectors may also be used for high-level protein expression.

Routine cloning, subcloning, and propagation of polynucleotides can be achieved using the multifunctional pBLUESCRIPT vector (Stratagene, La Jolla Calif.) or pSPORT1 plasmid (Life Technologies). Introduction of a polynucleotide into the multiple cloning site of these vectors disrupts the lacZ gene and allows calorimetric screening for transformed bacteria. In addition, these vectors may be useful for in vitro transcription, dideoxy sequencing, single strand rescue with helper phage, and creation of nested deletions in the cloned sequence.

For long term production of recombinant proteins, the vector can be stably transformed into cell lines along with a selectable or visible marker gene on the same or on a separate vector. After transformation, cells are allowed to grow for about 1 to 2 days in enriched media and then are transferred to selective media. Selectable markers, antimetabolite, antibiotic, or herbicide resistance genes, confer resistance to the relevant selective agent and allow growth and recovery of cells which successfully express the introduced sequences. Resistant clones identified either by survival on selective media or by the expression of visible markers may be propagated using culture techniques. Visible markers are also used to estimate the amount of protein expressed by the introduced genes. Verification that the host cell contains the desired polynucleotide is based on DNA-DNA or DNA-RNA hybridizations or PCR amplification techniques.

The host cell may be chosen for its ability to modify a recombinant protein in a desired fashion. Such modifications include acetylation, carboxylation, glycosylation, phosphorylation, lipidation, acylation and the like. Post-translational processing which cleaves a “prepro” form may also be used to specify protein targeting, folding, and/or activity. Different host cells available from the ATCC (Manassas Va.) which have specific cellular machinery and characteristic mechanisms for post-translational activities may be chosen to ensure the correct modification and processing of the recombinant protein.

### Recovery of Proteins from Cell Culture

Heterologous moieties engineered into a vector for ease of purification include glutathione S-transferase (GST), 6xHis, FLAG, MYC, and the like. GST and 6xHis are purified using commercially available affinity matrices such as immobilized glutathione and metal-chelate resins, respectively. FLAG and MYC are purified using commercially available monoclonal and polyclonal antibodies. For ease of separation following purification, a sequence encoding a proteolytic cleavage site may be part of the vector located between the protein and the heterologous moiety. Methods for recombinant protein expression and purification are discussed in Ausubel (supra, unit 16) and are commercially available.

### Chemical Synthesis of Peptides

Proteins or portions thereof may be produced not only by recombinant methods, but also by using chemical methods well known in the art. Solid phase peptide synthesis may be carried out in a batchwise or continuous flow process which sequentially adds a-amino- and side chain-protected amino acid residues to an insoluble polymeric support via a linker group. A linker group such as methylamine-derivatized polyethylene glycol is attached to poly(styrene-co-divinylbenzene) to form the support resin. The amino acid residues are N-α-protected by acid labile Boc (t-butyloxycarbonyl) or base-labile Fmoc (9-fluorenylmethoxycarbonyl). The carboxyl group of the protected amino acid is coupled to the amine of the linker group to anchor the residue to the solid phase support resin. Trifluoroacetic acid or piperidine are used to remove the protecting group in the case of Boc or Fmoc, respectively. Each additional amino acid is added to the anchored residue using a coupling agent or pre-activated amino acid derivative, and the resin is washed. The full length peptide is synthesized by sequential deprotection, coupling of derivitized amino acids, and washing with dichloromethane and/or N, N-dimethylformamide. The peptide is cleaved between the peptide carboxy terminus and the linker group to yield a peptide acid or amide. These processes are described in the Novabiochem 1997/98 Catalog and Peptide Synthesis Handbook (San Diego Calif. pp. S1-S20). Automated synthesis may also be carried out on machines such as the ABI431A peptide synthesizer (ABI). A protein or portion thereof may be purified by preparative high performance liquid chromatography and its composition confirmed by amino acid analysis or by sequencing (Creighton (1984) Proteins. Structures and Molecular Properties, WH Freeman, New York N.Y.).

### Antibodies

Antibodies, or immunoglobulins (Ig), are components of immune response expressed on the surface of or secreted into the circulation by B cells. The prototypical antibody is a tetramer composed of two identical heavy polypeptide chains (H-chains) and two identical light polypeptide chains (L-chains) interlinked by disulfide bonds which binds and neutralizes foreign antigens. Based on their H-chain, antibodies are classified as IgA, IgD, IgE, IgG or IgM. The most common class, IgG, is tetrameric while other classes are variants or multimers of the basic structure.

Antibodies are described in terms of their two main functional domains. Antigen recognition is mediated by the Fab (antigen binding fragment) region of the antibody, while effector functions are mediated by the Fc (crystallizable fragment) region. The binding of antibody to antigen triggers destruction of the antigen by phagocytic white blood cells such as macrophages and neutrophils. These cells express surface Fc receptors that specifically bind to the Fc region of the antibody and allow the phagocytic cells to destroy antibody-bound antigen. Fc receptors are single-pass transmembrane glycoproteins containing about 350 amino acids whose extracellular portion typically contains two or three Ig domains (Sears et al. (1990) J Immunol 144:371-378).

### Preparation and Screening of Antibodies

Various hosts including mice, rats, rabbits, goats, llamas, camels, and human cell lines may be immunized by injection with an antigenic determinant. Adjuvants such as Freund's, mineral gels, and surface active substances such as lysolecithin, pluronic polyols, polyanions, peptides, oil emulsions, keyhole limpet hemacyanin (KLH; Sigma-Aldrich, St. Louis Mo.), and dinitrophenol may be used to increase immunological response. In humans, BCG (bacilli Calmette-Guerin) and Corynebacterium parvum are preferable. The antigenic determinant may be an oligopeptide, peptide, or protein. When the amount of antigenic determinant allows immunization to be repeated, specific polyclonal antibody with high affinity can be obtained (Klinman and Press (1975) Transplant Rev 24:41-83). Oligopepetides which may contain between about five and about fifteen amino acids identical to a portion of the endogenous protein may be fused with proteins such as KLH in order to produce antibodies to the chimeric molecule.

Monoclonal antibodies may be prepared using any technique which provides for the production of antibodies by continuous cell lines in culture. These include the hybridoma technique, the human B-cell hybridoma technique, and the EBV-hybridoma technique (Kohler et al (1975) Nature 256:495497; Kozbor et al (1985) J Immunol Methods 81:3142; Cote et al (1983) Proc Natl Acad Sci 80:2026-2030; and Cole et al (1984) Mol Cell Biol 62:109-120).

“Chimeric antibodies” may be produced by techniques such as splicing of mouse antibody genes to human antibody genes to obtain a molecule with antigen specificity and biological activity (Morrison et al. (1984) Proc Natl Acad Sci 81:6851-6855; Neuberger et al. (1984) Nature 312:604-608; and Takeda et al. (1985) Nature 314:452454). Alternatively, techniques described for antibody production may be adapted, using methods known in the art, to produce specific, single chain antibodies. Antibodies with related specificity, but of distinct idiotypic composition, may be generated by chain shuffling from random combinatorial immunoglobulin libraries (Burton (1991) Proc Natl Acad Sci 88:10134-10137). Antibody fragments which contain specific binding sites for an antigenic determiinant may also be produced. For example, such fragments include, but are not limited to, F(ab′)2 fragments produced by pepsin digestion of the antibody molecule and Fab fragments generated by reducing the disulfide bridges of the F(ab′)2 fragments. Alternatively, Fab expression libraries may be constructed to allow rapid and easy identification of monoclonal Fab fragments with the desired specificity (Huse et al (1989) Science 246:1275-1281).

Antibodies may also be produced by inducing production in the lymphocyte population or by screening immunoglobulin libraries or panels of highly specific binding reagents as disclosed in Orlandi et al. (1989; Proc Natl Acad Sci 86:3833-3837) or Winter et al. (1991; Nature 349:293-299). A protein may be used in screening assays of phagemid or B-lymphocyte immunoglobulin libraries to identify antibodies having a desired specificity. Numerous protocols for competitive binding or immunoassays using either polyclonal or monoclonal antibodies with established specificities are well known in the art.

### Antibody Specificity

Various methods such as Scatchard analysis combined with radioimmunoassay techniques may be used to assess the affinity of particular antibodies for a protein. Affinity is expressed as an association constant, Kₐ, which is defined as the molar concentration of protein-antibody complex divided by the molar concentrations of free antigen and free antibody under equilibrium conditions. The Kₐ determined for a preparation of polyclonal antibodies, which are heterogeneous in their affinities for multiple antigenic determinants, represents the average affinity, or avidity, of the antibodies. The Kₐ determined for a preparation of monoclonal antibodies, which are specific for a particular antigenic determinant, represents a true measure of affinity. High-affinity antibody preparations with Kₐ ranging from about 10⁹ to 10¹² L/mole are preferred for use in immunoassays in which the protein-antibody complex must withstand rigorous manipulations. Low-affinity antibody preparations with Kₐ ranging from about 10⁶ to 10⁷ L/mole are preferred for use in immunopurification and similar procedures which ultimately require dissociation of the protein, preferably in active form, from the antibody (Catty (1988) Antibodies, Volume I: A Practical Approach, IRL Press, Washington DC; Liddell and Cryer (1991) A Practical Guide to Monoclonal Antibodies, John Wiley & Sons, New York N.Y.).

The titer and avidity of polyclonal antibody preparations may be further evaluated to determine the quality and suitability of such preparations for certain downstream applications. For example, a polyclonal antibody preparation containing about 5-10 mg specific antibody/mil, is generally employed in procedures requiring precipitation of protein-antibody complexes. Procedures for making antibodies, evaluating antibody specificity, titer, and avidity, and guidelines for antibody quality and usage in various applications, are widely available (Catty, supra; Ausubel (sura pp. 11.1-11.31)

### Labeling of Molecules for Assay

A wide variety of reporter molecules and conjugation techniques are known by those skilled in the art and may be used in various nucleic acid, amino acid, and antibody assays. Synthesis of labeled molecules may be achieved using commercially available kits (Promega, Madison WI) for incorporation of a labeled nucleotide such as ³²P-dCTP (APB), Cy3-dCTP or Cy5-dCTP (APB), or amino acid such as ³⁵S-methionine (APB). Nucleotides and amino acids may be directly labeled with a variety of substances including fluorescent, chemiluminescent, or chromogenic agents, and the like, by chemical conjugation to amines, thiols and other groups present in the molecules using reagents such as BIODIPY or FITC (Molecular Probes, Eugene Oreg.).

### Diagnostics

### Nucleic Acid Assays

The polynucleotides, fragments, oligonucleotides, complementary RNA and DNA molecules, and PNAs may be used to detect and quantify differential gene expression for diagnostic purposes. Disorders of the prostate and gastrointestinal system associated with expression of SEQ ID NO:2 specifically include benign prostatic hyperplasia, congenital enterokinase deficiency, pancreatitis, ulcerative colitis and cancers, particularly adenocarcinomas and sarcomas, of the prostate, pancreas, esophagus, stomach, small intestine, large intestine, and colon. The diagnostic assay may use hybridization or quantitative PCR to compare gene expression in a biological sample from a patient to standard samples in order to detect differential gene expression. Qualitative and quantitative methods for this comparison are commercially available and well known in the art.

For example, the polynucleotide or probe may be labeled by standard methods and added to a biological sample from a patient under conditions for the formation of hybridization complexes. After an incubation period, the sample is washed and the amount of label (or signal) associated with hybridization complexes, is quantified and compared with a standard value. If complex formation in the patient sample is significantly altered (higher or lower) in comparison to either a normal or disease standard, then differential expression indicates the presence of a disorder.

In order to provide standards for establishing differential expression, normal and diseased tissue expression profiles are established. This is accomplished by combining a sample taken from normal subjects, either animal or human, with a polynucleotide under conditions for hybridization to occur. Standard hybridization complexes may be quantified by comparing the values obtained using normal subjects with values from an experiment in which a known amount of a purified sequence is used. Standard values obtained in this manner may be compared with values obtained from samples from patients who were diagnosed with a particular condition, disease, or disorder. Deviation from standard values toward those associated with a particular disorder is used to diagnose that disorder.

Such assays may also be used to evaluate the efficacy of a particular therapeutic treatment regimen in animal studies or in clinical trials or to monitor the treatment of an individual patient. Once the presence of a condition is established and a treatment protocol is initiated, diagnostic assays may be repeated on a regular basis to determine if the level of expression in the patient begins to approximate that which is observed in a normal subject. The results obtained from successive assays may be used to show the efficacy of treatment over a period ranging from several days to years.

### Proteomic/Immunological Assays

Detection and quantification of a protein using either labeled amino acids or antibodies which specifically bind the protein are known in the art. Examples of such techniques include two-dimensional polyacrylamide gel electrophoresis, enzyme-linked immunosorbent assays (ELISAs), radioimmunoassays (RIAs), fluorescence-activated cell sorting (FACS) and antibody arrays. Such immunoassays typically involve the measurement of complex formation between the protein and its specific antibody. A two-site, monoclonal-based immunoassay utilizing antibodies reactive to two non-interfering epitopes is preferred, but a competitive binding assay may be employed (Coligan et al. (1997) Current Protocols in Immunology, Wiley-Interscience, New York N.Y.; Pound (1998) Immunochemical Protocols, Humana Press, Totowa N.J.). These assays and their quantitation against purified, labeled standards are well known in the art (Ausubel, supra, units 10.1-10.6).

Normal or standard values for protein expression are established by combining body fluids or cell extracts taken from a normal mammalian or human subject with specific antibodies to a protein under conditions for complex formation. Standard values for complex formation in normal and diseased tissues are established by various methods, often photometric means. Then complex formation as it is expressed in a subject sample is compared with the standard values. Deviation from the normal standard and toward the diseased standard provides parameters for disease diagnosis or prognosis while deviation away from the diseased and toward the normal standard may be used to evaluate treatment efficacy.

Proteomic and immunological methods are also useful for showing differential gene expression associated with the diagnosis of disorders of the prostate and gastrointestinal system including benign prostatic hyperplasia, congenital enterokinase deficiency, pancreatitis, ulcerative colitis and cancers, particularly adenocarcinomas and sarcomas, of the prostate, pancreas, esophagus, stomach, small intestine, large intestine, and colon.

### Therapeutics

HUPAP, a serine protease which appears to function in the prostate gland, shares chemical and structural homology with bovine enterokinase, human pancreatic kallikrein, and African rat renal kallikrein. In addition, northern analysis shows that four cDNA libraries containing HUPAP transcripts are from prostate, and three of these four were from patients with prostate cancer. HUPAP expression was also found in spinal cord, colon tissue, and pancreatic islet cells. Thus, HUPAP expression appears to be most closely associated with disorders of the prostate and gastrointestinal system.

The protease activity of HUPAP may activate certain digestive enzymes. Therefore, in one embodiment, HUPAP, an agonist which specifically binds HAPAP, or a vector capable of expressing HUPAP or a portion or derivative thereof, may be administered to a subject in need of such treatment having a gastrointestinal disorder such as congenital enterokinase deficiency or pancreatitis.

In another embodiment, antibodies, antagonists, or inhibitors of HUPAP or a vector expressing antisense of the polynucleotide encoding HUPAP may be used to suppress excessive cell proliferation. Such antibodies, antagonists, inhibitors of HUPAP or vectors may be administered to a subject in need of such treatment to suppress cell proliferation in disorders including benign prostatic hyperplasia, ulcerative colitis, and cancers such as adenocarcinomas and sarcomas of the prostate, pancreas, small intestine, large intestine, stomach, esophagus, and colon. In one aspect, antibodies which are specific for HUPAP may also be used to deliver a pharmaceutical agent to cells or tissue which express HUPAP.

Any of the compositions containing the polynucleotides, protein, or antibodies may be administered in combination with other therapeutic agents. Selection of the agents for use in combination therapy may be made by one of ordinary skill in the art according to conventional pharmaceutical principles. A combination of therapeutic agents may act synergistically to affect treatment of a particular cancer at a lower dosage of each agent alone.

### Modification of Gene Expression Using Nucleic Acids

Gene expression may be modified by designing complementary or antisense molecules (DNA, RNA, or PNA) to the control, 5′, 3′, or other regulatory regions of the gene encoding HUPAP. Oligonucleotides designed to inhibit transcription initiation are preferred. Similarly, inhibition can be achieved using triple helix base-pairing which inhibits the binding of polymerases, transcription factors, or regulatory molecules (Gee et al. In: Huber and Carr (1994) Molecular and Immunologic Approaches, Futura Publishing, Mt. Kisco N.Y., pp. 163-177). A complementary molecule may also be designed to block translation by preventing binding between ribosomes and mRNA. In one alternative, a library or plurality of polynucleotides may be screened to identify those which specifically bind a regulatory, nontranslated sequence.

Ribozymes, enzymatic RNA molecules, may also be used to catalyze the specific cleavage of RNA. The mechanism of ribozyme action involves sequence-specific hybridization of the ribozyme molecule to complementary target RNA followed by endonucleolytic cleavage at sites such as GUA, GUU, and GUC. Once such sites are identified, an oligonucleotide with the same sequence may be evaluated for secondary structural features which would render the oligonucleotide inoperable. The suitability of candidate targets may also be evaluated by testing their hybridization with complementary oligonucleotides using ribonuclease protection assays.

Complementary nucleic acids and ribozymes of the invention may be prepared via recombinant expression, in vitro or in vivo, or using solid phase phosphoramidite chemical synthesis. In addition, RNA molecules may be modified to increase intracellular stability and half-life by addition of flanking sequences at the 5′ and/or 3′ ends of the molecule or by the use of phosphorothioate or 2′ O-methyl rather than phosphodiesterase linkages within the backbone of the molecule. Modification is inherent in the production of PNAs and can be extended to other nucleic acid molecules. Either the inclusion of nontraditional bases such as inosine, queosine, and wybutosine, or the modification of adenine, cytidine, guanine, thymine, and uridine with acetyl-, methyl-, thio- groups renders the molecule less available to endogenous endonucleases.

### Screening and Purification Assays

The polynucleotide encoding HUPAP may be used to screen a plurality or a library of molecules or compounds for specific binding affinity. The libraries may be aptamers, DNA molecules, RNA molecules, PNAs, peptides, proteins such as transcription factors, enhancers, or repressors, and other ligands which regulate the activity, replication, transcription, or translation of the endogenous gene. The assay involves combining a polynucleotide with a plurality of molecules or compounds under conditions allowing specific binding, and detecting specific binding to identify at least one molecule which specifically binds the single-stranded or double-stranded molecule.

In one embodiment, the polynucleotide of the invention may be incubated with a plurality of purified molecules or compounds and binding activity determined by methods well known in the art, e.g., a gel-retardation assay (U.S. Pat. No. 6,010,849) or a commercially available reticulocyte lysate transcriptional assay. In another embodiment, the polynucleotide may be incubated with nuclear extracts from biopsied and/or cultured cells and tissues. Specific binding between the polynucleotide and a molecule or compound in the nuclear extract is initially determined by gel shift assay and may be later confirmed by recovering and raising antibodies against that molecule or compound. When these antibodies are added into the assay, they cause a supershift in the gel-retardation assay.

In another embodiment, the polynucleotide may be used to purify a molecule or compound using affinity chromatography methods well known in the art. In one embodiment, the polynucleotide is chemically reacted with cyanogen bromide groups on a polymeric resin or gel. Then a sample is passed over and reacts with or binds to the polynucleotide. The molecule or compound which is bound to the polynucleotide may be released from the polynucleotide by increasing the salt concentration of the flow-through medium and collected.

In a further embodiment, HUPAP or a portion thereof may be used to purify a ligand from a sample. A method for using a protein or a portion thereof to purify a ligand would involve combining the protein or a portion thereof with a sample under conditions to allow specific binding, detecting specific binding between the protein and ligand, recovering the bound protein, and using a chaotropic agent to separate the protein from the purified ligand.

In a preferred embodiment, HUPAP may be used to screen a plurality of molecules or compounds in any of a variety of screening assays. The portion of the protein employed in such screening may be free in solution, affixed to an abiotic or biotic substrate (e.g. borne on a cell surface), or located intracellularly. For example, in one method, viable or fixed prokaryotic host cells that are stably transformed with recombinant nucleic acids that have expressed and positioned a peptide on their cell surface can be used in screening assays. The cells are screened against a plurality or libraries of ligands, and the specificity of binding or formation of complexes between the expressed protein and the ligand can be measured. Depending on the particular kind of molecules or compounds being screened, the assay may be used to identify DNA molecules, RNA molecules, peptide nucleic acids, peptides, proteins, mimetics, agonists, antagonists, antibodies, immunoglobulins, inhibitors, and drugs of any kind which specifically binds the protein.

In one aspect, drug screening may be accomplished using methods for high throughput. In the method of PCT application WO84/03564, large numbers of test compounds are synthesized on a solid substrate and reacted with HUPAP. The substrate is washed, and bound HUPAP is detected by methods well known in the art. In an alternative, purified HUPAP can be coated directly onto plates or immobilized by non-neutralizing antibodies for use in the technique.

In another aspect, this invention comtemplates a method for high throughput screening using very small assay volumes and very small amounts of test compound as described in USPN 5,876,946, incorporated herein by reference. This method is used to screen large numbers of molecules and compounds via specific binding. In yet another aspect, this invention also contemplates the use of competitive drug screening assays in which neutralizing antibodies capable of binding the protein specifically compete with a test compound capable of binding to the protein. Molecules or compounds identified by screening methods may be used in a model system to evaluate their toxicity, diagnostic, or therapeutic potential.

### Pharmaceutical Compositions

An additional embodiment of the invention relates to the administration of a pharmaceutical composition for any of the therapeutic effects discussed above. Such pharmaceutical compositions may contain HUPAP, antibodies specifically binding HUPAP, mimetics, agonists, antagonists, or inhibitors of HUPAP and a sterile, biocompatible pharmaceutical carrier such as saline, buffered saline, dextrose, or water or excipients and auxiliaries which facilitate processing of the active compounds into preparations which can be used pharmaceutically. The compositions may be administered to a patient alone or in combination with other agents, drugs, or hormones.

The pharmaceutical compositions of the present invention may be manufactured by means of conventional mixing, dissolving, granulating, dragee-making, levigating, emulsifying, encapsulating, entrapping, or lyophilizing processes. Further details on techniques for formulation and administration may be found in the latest edition of Remington's Pharmaceutical Sciences (Mack Publishing, Easton Pa.).

The pharmaceutical compositions utilized in this invention may be administered by any number of routes including, but not limited to, oral, intravenous, intramuscular, intra-arterial, intramedullary, intrathecal, intraventricular, transdermal, subcutaneous, intraperitoneal, intranasal, enteral, topical, sublingual, or rectal. Pharmaceutical compositions for oral administration can be formulated using carriers in dosages suitable for oral administration. Such carriers enable the pharmaceutical compositions to be formulated as tablets, pills, dragees, capsules, liquids, gels, syrups, slurries, suspensions, and the like, for ingestion by the patient. Excipients include carbohydrate or protein fillers, such as lactose, sucrose, mannitol, or sorbitol; starch from corn, wheat, rice, potato, or other plants; cellulose, such as methyl cellulose, hydroxypropylmethyl-cellulose, or sodium carboxymethylcellulose; gums including arabic and tragacanth; and proteins such as gelatin and collagen.

Disintegrating or solubilizing agents such as the cross-linked polyvinyl pyrrolidone, agar, alginic acid, or sodium alginate may be added. Dragee cores may be coated with concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of active compound or dosage.

Pharmaceutical preparations which can be used orally include push-fit capsules made of gelatin, as well as soft, sealed capsules made of gelatin and a coating, such as glycerol or sorbitol. Push-fit capsules can contain active ingredients mixed with a filler or binders, such as lactose or starches, lubricants, such as talc or magnesium stearate, and, optionally, stabilizers. In soft capsules, the active compounds may be dissolved or suspended in liquids such as fatty oils or liquid polyethylene glycol with or without stabilizers.

Pharmaceutical formulations for parenteral administration may be formulated in aqueous solutions, preferably in physiologically compatible buffers such as Hanks' solution, Ringer's solution, or physiologically buffered saline. Aqueous injection suspensions may contain substances which increase the viscosity of the suspension, such as sodium carboxymethyl cellulose, sorbitol, or dextran. Additionally, suspensions of the active compounds may be prepared as oily injection suspensions. Lipophilic solvents or vehicles include fatty oils such as sesame oil, or synthetic fatty acid esters, such as ethyl oleate or triglycerides, or liposomes. Optionally, the suspension may also contain stabilizers or agents which increase the solubility of the compounds to allow for the preparation of highly concentrated solutions.

For topical or nasal administration, penetrants well known in the art are used in the formulation.

The pharmaceutical composition may be provided as a salt and can be formed with many acids, including but not limited to, hydrochloric, sulfuric, acetic, lactic, tartaric, malic, succinic, etc. Salts tend to be more soluble in aqueous or other protonic solvents than are the corresponding free base forms. In other cases, the preferred preparation may be a lyophilized powder which may contain any or all of the following: 1-50 mM histidine, 0.1%-2% sucrose, and 2-7% mannitol, at a pH range of 4.5 to 5.5, that is combined with buffer prior to use.

After pharmaceutical compositions have been prepared, they can be placed in a container and abeled for treatment of an indicated condition. For administration of HUPAP, such labeling would include amount, frequency, and method of administration.

Pharmaceutical compositions for use in the invention include compositions wherein the active ingredients are contained in an effective amount to achieve the intended purpose. The determination of an effective dose is well within the capability of those skilled in the art. For any compound, the therapeutically effective dose can be estimated initially either in cell culture assays, e.g., of neoplastic cells, or in animal models, usually mice, rabbits, dogs, or pigs. The animal model may also be used to determine the concentration range, useful dose, and route of administration for humans.

A therapeutically effective dose refers to that amount of active ingredient, for example HUPAP or fragments thereof, antibodies of HUPAP, agonists, antagonists or inhibitors of HUPAP, which ameliorates the symptoms or condition. Therapeutic efficacy and toxicity may be determined by standard pharmaceutical procedures in cell cultures or experimental animals, e.g., ED50 (the dose therapeutically effective in 50% of the population) and LD50 (the dose lethal to 50% of the population). The dose ratio between therapeutic and toxic effects is the therapeutic index, and it can be expressed as the ratio, LD50/ED50.

Pharmaceutical compositions which exhibit large therapeutic indices are preferred. The data obtained from cell culture assays and animal studies is used in formulating a range of dosage for human use. The dosage contained in such compositions is within a range of circulating concentrations that include the ED50 with little or no toxicity. The dosage varies within this range depending upon the dosage form employed, sensitivity of the patient, and the route of administration.

The exact dosage will be determined by the practitioner, in light of factors related to the subject in need of the treatment. Dosage and administration are adjusted to provide sufficient levels of the active moiety or to maintain the desired effect. Factors which may be taken into account include the severity of the disease state, general health of the subject, age, weight, and gender of the subject, diet, time and frequency of administration, drug combination(s), reaction sensitivities, and tolerance/response to therapy. Long-acting pharmaceutical compositions may be administered every 3 to 4 days, every week, or once every two weeks depending on half-life and clearance rate of the particular formulation.

Normal dosage amounts may vary from 0.1 μg, up to a total dose of about 1 g, depending upon the route of administration. Guidance as to particular dosages and methods of delivery is provided in the literature and generally available to practitioners in the art. Those skilled in the art will employ different formulations for nucleotides than for proteins or their inhibitors. Similarly, delivery of polynucleotides or proteins will be specific to particular cells, conditions, locations, etc.

### Model Systems

Animal models may be used as bioassays where they exhibit a phenotypic response similar to that of humans and where exposure conditions are relevant to human exposures. Mammals are the most common models, and most infectious agent, cancer, drug, and toxicity studies are performed on rodents such as rats or mice because of low cost, availability, lifespan, reproductive potential, and abundant reference literature. Inbred and outbred rodent strains provide a convenient model for investigation of the physiological consequences of under- or over-expression of genes of interest and for the development of methods for diagnosis and treatment of diseases. A mammal inbred to over-express a particular gene (for example, secreted in milk) may also serve as a convenient source of the protein expressed by that gene.

### Toxicology

Toxicology is the study of the effects of agents on living systems. The majority of toxicity studies are performed on rats or mice. Observation of qualitative and quantitative changes in physiology, behavior, homeostatic processes, and lethality in the rats or mice are used to generate a toxicity profile and to assess potential consequences on human health following exposure to the agent.

Genetic toxicology identifies and analyzes the effect of an agent on the rate of endogenous, spontaneous, and induced genetic mutations. Genotoxic agents usually have common chemical or physical properties that facilitate interaction with nucleic acids and are most harmful when chromosomal aberrations are transmitted to progeny. Toxicological studies may identify agents that increase the frequency of structural or functional abnormalities in the tissues of the progeny if administered to either parent before conception, to the mother during pregnancy, or to the developing organism. Mice and rats are most frequently used in these tests because their short reproductive cycle allows the production of the numbers of organisms needed to satisfy statistical requirements.

Acute toxicity tests are based on a single administration of an agent to the subject to determine the symptomology or lethality of the agent. Three experiments are conducted: 1) an initial dose-range-finding experiment, 2) an experiment to narrow the range of effective doses, and 3) a final experiment for establishing the dose-response curve.

Subchronic toxicity tests are based on the repeated administration of an agent. Rat and dog are commonly used in these studies to provide data from species in different families. With the exception of carcinogenesis, there is considerable evidence that daily administration of an agent at high-dose concentrations for periods of three to four months will reveal most forms of toxicity in adult animals.

Chronic toxicity tests, with a duration of a year or more, are used to demonstrate either the absence of toxicity or the carcinogenic potential of an agent. When studies are conducted on rats, a minimum of three test groups plus one control group are used, and animals are examined and monitored at the outset and at intervals throughout the experiment.

### Transgenic Animal Models

Transgenic rodents that over-express or under-express a gene of interest may be inbred and used to model human diseases or to test therapeutic or toxic agents. (See, e.g., U.S. Pat. No. 5,175,383 and U.S. Pat. No. 5,767,337.) In some cases, the introduced gene may be activated at a specific time in a specific tissue type during fetal or postnatal development. Expression of the transgene is monitored by analysis of phenotype, of tissue-specific mRNA expression, or of serum and tissue protein levels in transgenic animals before, during, and after challenge with experimental drug therapies.

### Embryonic Stem Cells

Embryonic (ES) stem cells isolated from rodent embryos retain the potential to form embryonic tissues. When ES cells are placed inside a carrier embryo, they resume normal development and contribute to tissues of the live-born animal. ES cells are the preferred cells used in the creation of experimental knockout and knockin rodent strains. Mouse ES cells, such as the mouse 129/SvJ cell line, are derived from the early mouse embryo and are grown under culture conditions well known in the art. Vectors used to produce a transgenic strain contain a disease gene candidate and a marker gen, the latter serves to identify the presence of the introduced disease gene. The vector is transformed into ES cells by methods well known in the art, and transformed ES cells are identified and microinjected into mouse cell blastocysts such as those from the C57BL/6 mouse strain. The blastocysts are surgically transferred to pseudopregnant dams, and the resulting chimeric progeny are genotyped and bred to produce heterozygous or homozygous strains.

ES cells derived from human blastocysts may be manipulated in vitro to differentiate into at least eight separate cell lineages. These lineages are used to study the differentiation of various cell types and tissues in vitro, and they include endoderm, mesoderm, and ectodermal cell types which differentiate into, for example, neural cells, hematopoietic lineages, and cardiomyocytes. Knockout Analysis

In gene knockout analysis, a region of a gene is enzymatically modified to include a non-mammalian gene such as the neomycin phosphotransferase gene (neo; Capecchi (1989) Science 244:1288-1292). The modified gene is transformed into cultured ES cells and integrates into the endogenous genome by homologous recombination. The inserted sequence disrupts transcription and translation of the endogenous gene. Transformed cells are injected into rodent blastulae, and the blastulae are implanted into pseudopregnant dams. Transgenic progeny are crossbred to obtain homozygous inbred lines which lack a functional copy of the mammalian gene. In one example, the mammalian gene is a human gene.

### Knockin Analysis

ES cells can be used to create knockin humanized animals (pigs) or transgenic animal models (mice or rats) of human diseases. With knockin technology, a region of a human gene is injected into animal ES cells, and the human sequence integrates into the animal cell genome. Transformed cells are injected into blastulae and the blastulae are implanted as described above. Transgenic progeny or inbred lines are studied and treated with potential pharmaceutical agents to obtain information on treatment of the analogous human condition. These methods have been used to model several human diseases.

### Non-Human Primate Model

The field of animal testing deals with data and methodology from basic sciences such as physiology, genetics, chemistry, pharmacology and statistics. These data are paramount in evaluating the effects of therapeutic agents on non-human primates as they can be related to human health. Monkeys are used as human surrogates in vaccine and drug evaluations, and their responses are relevant to human exposures under similar conditions. Cynomolgus and Rhesus monkeys (Macaca fascicularis and Macaca mulatta, respectively) and Common Marmosets (Callithrix jacchus) are the most common non-human primates (NHPs) used in these investigations. Since great cost is associated with developing and maintaining a colony of NHPs, early research and toxicological studies are usually carried out in rodent models. In studies using behavioral measures such as drug addiction, NHPs are the first choice test animal. In addition, NHPs and individual humans exhibit differential sensitivities to many drugs and toxins and can be classified as a range of phenotypes from “extensive metabolizers” to “poor metabolizers” of these agents.

In additional embodiments, HUPAP, the polynucleotides which encode HUPAP, and antibodies which specifically bind HUPAP may be used in any molecular biology techniques that have yet to be developed, provided the new techniques rely on properties of polynucleotides that are currently known, including, but not limited to, such properties as the triplet genetic code and specific base pair interactions. The examples below are provided to illustrate the invention and are not included for the purpose of limiting the invention.

### EXAMPLES

I cDNA Library Construction

The SCORNOT01 cDNA library was constructed from normal spinal cord removed from a 71 year old, Caucasian male (lot #RA95-04-0255; obtained from the Keystone Skin Bank, International Institute for Advanced Medicine, Exton Pa.). The tissue was flash frozen, ground in a mortar and pestle, and lysed immediately in a buffer containing guanidinium isothiocyanate. The lysate was extracted once with acid phenol, pH 4.0, once with phenol chloroform, pH 8.0, and then centrifuged over a CsCl cushion using an SW28 rotor in a L8-70M ultracentrifuge (Beckman Coulter, Fullerton Calif.). The RNA was precipitated from 0.3 M sodium acetate using 2.5 volumes of ethanol, resuspended in water and DNAse treated for 15 min at 37 C. The poly A+RNA was isolated with the OLIGOTEX kit (Qiagen, Chatsworth Calif.) and used to construct the cDNA library.

The RNA was handled according to the recommended protocols in the SUPERSCRIPT plasmid system (Life Technologies). cDNAs were fractionated on a SEPHAROSE CL4B column (APB), and those cDNAs exceeding 400 bp were ligated into pSPORT I plasmid (Life Technologies). The plasmid was subsequently transformed into DH5α competent cells (Life Technologies).

II Isolation and Sequencing of cDNA Clones

Plasmid DNA was purified using the MINIPREP Kit (AGTC Corporation, Gaithersburg Md.), a 96-well block kit with reagents for 960 purifications. The recommended protocol included with the kit was employed except for the following changes. Each of the 96 wells was filled with only 1 ml of sterile TERRIFIC BROTH (BD Biosciences, San Jose Calif.) with carbenicillin at 25 mg/L and glycerol at 0.4%. The bacteria were cultured for 24 hours and lysed with 60 μl of lysis buffer. The lysate was centrifuged @2900 rpm for 5 min in a GS-6R (Beckman Coulter) before the contents of the block were added to the primary filter plate. An optional step of adding isopropanol was not routinely performed. After the last step in the protocol, samples were transferred to a 96-well block for storage.

The cDNAs were prepared using a MICROLAB 2200 (Reno Nev.) in combination with four DNA ENGINE thermal cyclers ( MJ Research). cDNAs were sequenced by the method of Sanger and Coulson (1975; J Mol Biol 94:441f), using ABI PRISM 377 or 373 DNA sequencing systems (ABI), and reading frame was determined.

III Homology Searching of Polynucleotides and Their Deduced Proteins

SEQ ID NOs: 1 and 2 were used as query sequences against databases such as GenBank, SwissProt, BLOCKS, and Pima II. These databases which contain previously identified and annotated sequences were searched for regions of homology (similarity) using BLAST, which stands for Basic Local Alignment Search Tool (Altschul (1993) J Mol Evol 36:290-300; Altschul et al. (1990) J Mol Biol 215:403-10).

BLAST produces alignments of both nucleic acid and amino acid sequences to determine sequence similarity. Because of the local nature of the alignments, BLAST is especially useful in determining exact matches or in identifying homologs which may be of prokaryotic (bacterial) or eukaryotic (animal, fungal or plant) origin. Other algorithms such as the one described in Smith and Smith (1992 Protein Engineering 5:35-51), incorporated herein by reference, can be used when dealing with primary sequence patterns and secondary structure gap penalties. As disclosed in this application, the sequences in the Sequence Listing have a minimum length of at lease 49 nucleotides, and no more than 12% uncalled bases (where N is recorded rather than A, C, G, or T).

The BLAST approach, as detailed in Karlin and Altschul (1993; Proc Natl Acad Sci 90:5873-7) and incorporated herein by reference, searches matches between a query sequence and a database sequence, to evaluate the statistical significance of any matches found, and to report only those matches which satisfy the user-selected threshold of significance. In this application, threshold was set at 10-25 for nucleotides and 10-14 for peptides.

The polynucleotides were searched against the GenBank databases for pri=primate, rod=rodent, and mam=mammalian sequences, and deduced amino acid sequences from the same clones are searched against GenBank functional protein databases, mamp=mammalian, vrtp=vertebrate and eukp=eukaryote, for homology. The relevant database for a particular match were reported as a GIxxx+/−p (where xxx is for pri, rod, etc. and p, if found, refers to protein database). The product score =(% nucleotide or amino acid identity [between the query and reference sequences] in Blast multiplied by the % maximum possible BLAST score [based on the lengths of query and reference sequences]) divided by 100. Where an Incyte Clone was homologous to several sequences, up to five matches were provided with their relevant scores. In an analogy to the hybridization procedures used in the laboratory, a conservative, electronic stringency was set at 70 (“exact” match), and the absolute cutoff for was set at 40 (1-2% error due to uncalled bases).

IV Northern Analysis

Northern analysis is a laboratory technique used to detect the presence of a transcript of a gene and involves the hybridization of a labeled polynucleotide to a membrane on which mRNAs from a particular cell type or tissue have been bound (Sambrook, supra).

Analogous computer techniques using BLAST (Altschul, 1993 and 1990, supra) are used to search for identical or related molecules in nucleotide databases such as the GenBank or LIFESEQ databases (Incyte Genomics). This analysis is much faster than multiple, membrane-based hybridizations. In addition, the sensitivity of the computer search can be modified to determine whether any particular match is categorized as exact or homologous.

The basis of the search is the product score which is defined as:

$$\frac{\% sequence identity \times \% maximum BLAST score}{100}$$

The product score takes into account both the degree of similarity between two sequences and the length of the sequence match. For example, with a product score of 40, the match will be exact within a 1-2% error; and at 70, the match will be exact. Homologous molecules are usually identified by selecting those which show product scores between 15 and 40, although lower scores may identify related molecules.

The results of northern analysis are reported as a list of libraries in which the transcript encoding HUPAP occurs. Abundance and percent abundance are also reported. Abundance directly reflects the number of times a particular transcript is represented in a cDNA library, and percent abundance is abundance divided by the total number of sequences examined in the cDNA library.

V Extension of HUPAP-Encoding Polynucleotides

The full length HUPAP-encoding polynucleotide (SEQ ID NO:2) is used to design oligonucleotide primers for extending a partial nucleotide sequence to full length or for obtaining 5′ or 3′, intron, or other control sequences from genomic libraries. One primer is synthesized to initiate extension in the antisense direction (XLR) and the other is synthesized to extend sequence in the sense direction (XLF). Primers are used to facilitate the extension of the known sequence “outward” generating amplicons containing new, unknown nucleotide sequence for the region of interest. The initial primers are designed from the cDNA using commercially available software to be 22-30 nucleotides in length, to have a GC content of 50% or more, and to anneal to the target sequence at temperatures about 68-72 C. Any stretch of nucleotides which would result in hairpin structures and primer-primer dimerizations is avoided.

The original, selected cDNA libraries, or a human genomic library are used to extend the sequence; the latter is most useful to obtain 5′ upstream regions. If more extension is desired, additional sets of primers are designed to further extend the known region.

By following the instructions for the XL-PCR kit (ABI) and thoroughly mixing the enzyme and reaction mix, high fidelity amplification is obtained. Beginning with 40 pmol of each primer and the recommended concentrations of all other components of the kit, PCR is performed using the DNA ENGINE thermal cycler (MJ Research) and the following parameters: Step 1, 94 C. for 1 min (initial denaturation); Step 2, 65 C. for 1 min; Step 3, 68 C. for 6 min; Step 4, 94 C. for 15 sec; Step 5, 65 C. for 1 min; Step 6, 68 C. for 7 min; Step 7, repeat step 4-6 for 15 additional cycles; Step 8, 94 C. for 15 sec; Step 9, 65 C. for 1 min; Step 10, 68 C. for 7:15 min; Step 11, repeat step 8-10 for 12 cycles; Step 12, 72 C. for 8 min; and Step 13, 4 C. (and holding).

A 5-10 μl aliquot of the reaction mixture is analyzed by electrophoresis on a low concentration (about 0.6-0.8%) agarose mini-gel to determine which reactions were successful in extending the sequence. Bands thought to contain the largest products are selected and removed from the gel. Further purification involves using a commercial gel extraction method such as QIAQUICK (Qiagen). After recovery of the DNA, Klenow enzyme is used to trim single-stranded, nucleotide overhangs creating blunt ends which facilitate religation and cloning.

After ethanol precipitation, the products are redissolved in 13 μl of ligation buffer, 1 μl T4-DNA ligase (15 units) and 1 μl T4 polynucleotide kinase are added, and the mixture is incubated at room temperature for 2-3 hours or overnight at 16 C. Competent E. coli cells (in 40 μl of media) are transformed with 3 μl of ligation mixture and cultured in 80 μl of SOC medium (Sambrook, supra). After incubation for one hour at 37 C., the whole transformation mixture is plated on Luria Bertani (LB)-agar (Sambrook, supra) containing 2×carbenicillin (2×Carb). The following day, several colonies are randomly picked from each plate and cultured in 150 μl of liquid LB/2×Carb medium placed in an individual well of a commercially-available, sterile 96-well microtiter plate. The following day, 5 μl of each overnight culture is transferred into a non-sterile 96-well plate and after dilution 1:10 with water, 5 μl of each sample is transferred into a well on a PCR plate..

For PCR amplification, 18,μl of concentrated PCR reaction mix (3.3×) containing 4 units of rTth DNA polymerase, a vector primer, and one or both of the gene specific primers used for the extension reaction are added to each well. Amplification is performed using the following conditions: Step 1, 94 C. for 60 sec; Step 2, 94 C. for 20 sec; Step 3, 55 C. for 30 sec; Step 4, 72 C. for 90 sec; Step 5, repeat steps 2-4 for an additional 29 cycles; Step 6, 72 C. for 180 sec; and Step 7, 4 C. (and holding).

Aliquots of the PCR reactions are run on agarose gels together with molecular weight markers. The sizes of the PCR products are compared to the original partial cDNAs, and clones are selected, ligated into plasmid, and sequenced using the methods and machines detailed above.

VI Labeling and Use of Hybridization Probes

Hybridization probes derived from SEQ ID NO:2 are employed to screen cDNAs, genomic DNAs, or mRNAs. Although the labeling of oligonucleotides, consisting of about 20 base-pairs, is specifically described, the same procedure is used with larger polynucleotide fragments. Oligonucleotides are designed using commercially available programs, such as those in LASERGENE software (DNASTAR), and labeled by combining 50 pmol of each oligomer and 250 μCi of [γ-³²p] adenosine triphosphate (APB) and T4 polynucleotide kinase (DuPont NEN Research Products, Boston Mass.). The labeled oligonucleotides are purified with SEPHADEX G-25 superfine resin column (APB). An aliquot containing 10⁷ counts per minute of each of the sense and antisense oligonucleotides is used in a typical membrane based hybridization analysis of human genomic DNA digested with one of the following endonucleases (Ase I, Bgl II, Eco RI, Pst I, Xba 1, or Pvu II; DuPont NEN Research Products).

The DNA from each digest is fractionated on a 0.7 percent agarose gel and transferred to NYTRAN PLUS membranes (Schleicher & Schuell, Durham N.H.). Hybridization is carried out for 16 hours at 40 C. To remove nonspecific signals, blots are sequentially washed at room temperature under increasingly stringent conditions up to 0.1×saline sodium citrate and 0.5% sodium dodecyl sulfate. XOMAT AR film (Eastman Kodak, Rochester N.Y.) is exposed to the blots, and hybridization patterns are compared.

VII Antisense Molecules

Antisense molecules to the HUPAP-encoding sequence, or any part thereof, is used to inhibit in vivo or in vitro expression of naturally occurring HUPAP. Although use of antisense oligonucleotides, comprising about 20 base-pairs, is specifically described, the same procedure is used with larger polynucleotide fragments. An oligonucleotide based on the coding sequences of HUPAP, as shown in FIGS. 1A and 1B, is used to inhibit expression of naturally occurring HUPAP. The complementary oligonucleotide is designed from the most unique 5′ sequence as shown in FIGS. 1A and 1B and used either to inhibit transcription by preventing promoter binding to the upstream nontranslated sequence or translation of an HUPAP-encoding transcript by preventing the ribosome from binding. Using a fragment of the signal and 5′ sequence of SEQ ID NO:2, an effective antisense oligonucleotide includes any 15-20 nucleotides spanning the region which translates into the signal or 5′ coding sequence of the protein as shown in FIGS. 1A and 1B.

VIII Expression of HUPAP

Expression of HUPAP is accomplished by subcloning the polynucleotides into expression vectors and transforming the vectors into host cells. In this case, the pSPORT1 vector, previously used for the generation of the cDNA library, is used to express HUPAP in E. coli. Upstream of the cloning site, this vector contains a promoter for β-galactosidase, followed by sequence containing the amino-terminal Met, and the subsequent seven residues of β-galactosidase. Immediately following these eight residues is a bacteriophage promoter useful for transcription and a linker containing a number of unique restriction sites.

Induction of an isolated, transformed bacterial strain with IPTG using standard methods produces a fusion protein which consists of the first eight residues of β-galactosidase, about 5 to 15 residues of linker, and the full length protein. The signal residues direct the secretion of HUPAP into the bacterial growth media which can be used directly in the following assay for activity.

IX Demonstration of HUPAP Activity

HUPAP's proteolytic activity can be determined by methods described by Christernsson et al. (1990, Eur J Biochem 194:755-763). Chemical substrates for proteolytic cleavage are found in human semen. Human seminal plasma is collected, and coagulated semen is washed free of soluble components. HUPAP is incubated with coagulated semen at 37 C. in a buffer consisting of 50 mmol/l TRIS-HCl pH 7.8, with 0.1 mol/l NaCl. After incubation periods of different durations (from 0 to 30 minutes), the samples are analyzed by SDS/PAGE. The resulting pattern of peptide fragments is quantitated and compared to a control sample handled identically but to which HUPAP is not added.

X Production of Specific Antibodies

Polyacrylamide gel electrophoresis or similar techniques are used to isolate HUPAP for immunization of hosts or host cells to produce antibodies using standard protocols.

Alternatively, the amino acid sequence of the protein is analyzed using readily available commercial software to determine regions of high immunogenicity. A peptide with high immunogenicity is cleaved, recombinantly-produced, or synthesized and used to raise antibodies by means known to those of skill in the art. Methods for selection of antigenic determinants such as those near the C-terminus or in hydrophilic regions are well described in the art (Ausubel, supra, Chap. 11).

Oligopeptides of about 15 residues in length are synthesized using an ABI 431A peptide synthesizer (ABI) using Fmoc chemistry and coupled to carriers such as BSA, thyroglobulin, or KLH (Sigma-Aldrich) by reaction with N-maleimidobenzoyl-N-hydroxysuccinimide ester to increase immunogenicity. The coupled peptide is then used to immunize the host. Rabbits are immunized with the oligopeptide-KLH complex in complete Freund's adjuvant. Resulting antisera are tested for antipeptide activity by binding the peptide to a substrate, blocking with 1% BSA, reacting with rabbit antisera, washing, and reacting with radio-iodinated goat anti-rabbit IgG.

XI Immunopurification Using Antibodies

Naturally occurring or recombinantly produced protein is purified by immunoaffinity chromatography using antibodies which specifically bind the protein. An immunoaffinity column is constructed by covalently coupling the antibody to CNBr-activated SEPHAROSE resin (APB). Media containing the protein is passed over the immunoaffinity column, and the column is washed using high ionic strength buffers in the presence of detergent to allow preferential absorbance of the protein. After coupling, the protein is eluted from the column using a buffer of pH 2-3 or a high concentration of urea or thiocyanate ion to disrupt antibody/protein binding, and the purified protein is collected.

XII Antibody Arrays

### Protein: Protein Interactions

In an alternative to yeast two hybrid system analysis of proteins, an antibody array can be used to study protein-protein interactions and phosphorylation. A variety of protein ligands are immobilized on a membrane using methods well known in the art. The array is incubated in the presence of cell lysate until protein:antibody complexes are formed. Proteins of interest are identified by exposing the membrane to an antibody specific to the protein of interest. In the alternative, a protein of interest is labeled with digoxigenin (DIG) and exposed to the membrane; then the membrane is exposed to anti-DIG antibody which reveals where the protein of interest forms a complex. The identity of the proteins with which the protein of interest interacts is determined by the position of the protein of interest on the membrane.

### Proteomic Profiles

Antibody arrays can also be used for high-throughput screening of recombinant antibodies. Bacteria containing antibody genes are robotically-picked and gridded at high density (up to 18,342 different double-spotted clones) on a filter. Up to 15 antigens at a time are used to screen for clones to identify those that express binding antibody fragments. As described by de Wildt et al. (2000; Nat Biotechnol 18:989-94), these antibody arrays can also be used to identify proteins which are differentially expressed in samples.

XIII Identification of Molecules Which Interact with HUPAP

HUPAP or biologically active portions thereof are labeled with ¹²⁵I Bolton-Hunter reagent (Bolton et al. (1973) Biochem J 133:529-539). Candidate molecules previously arrayed in the wells of a multi-well plate are incubated with the labeled HUPAP, washed and any wells with labeled HUPAP complex are assayed. Data obtained using different concentrations of HUPAP are used to calculate values for the number, affinity, and association of HUPAP with the candidate molecules.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the following claims.

What is claimed is:

1. A purified human prostate-associated protease comprising the amino acid sequence of SEQ ID NO:1 or portions thereof.

2. A purified antibody which binds specifically to the protein of claim 1.

3. The antibody of claim 2, wherein the antibody is selected from an intact immunoglobulin molecule, a polyclonal antibody, a monoclonal antibody, a chimeric antibody, a recombinant antibody, a humanized antibody, single chain antibodies, a Fab fragment, an F(ab′)₂ fragment, an Fv fragment; and an antibody-peptide fusion protein.

4. A method for preparing and purifying a polyclonal antibody with the specificity of the antibody of claim 2 comprising:
a) immunizing a animal with a protein of SEQ ID NO:1 under conditions to elicit an antibody response;
b) isolating animal antibodies;
c) attaching the protein to a substrate;
d) contacting the substrate with isolated antibodies under conditions to allow specific binding to the protein;
e) dissociating the antibodies from the protein, thereby obtaining purified polyclonal antibodies.

5. A polyclonal antibody produced by the method of claim 4.

6. A method for preparing a monoclonal antibody with the specificity of the antibody of claim 2 comprising:
a) immunizing a animal with a protein of SEQ ID NO:1 under conditions to elicit an antibody response;
b) isolating antibody-producing cells from the animal;
c) fusing the antibody-producing cells with immortalized cells in culture to form monoclonal antibody producing hybridoma cells;
d) culturing the hybridoma cells; and
e) isolating monoclonal antibodies from culture.

7. A monoclonal antibody produced by the method of claim 6.

8. A method for using an antibody to immunopurify a protein comprising:
a) attaching an antibody of claim 2 to a substrate,
b) exposing the antibody to a sample containing protein under conditions to allow antibody:protein complexes to form,
c) dissociating the protein from the complex, and
d) collecting the purified protein.

9. A method for using an antibody to detect expression of a protein in a sample, the method comprising:
a) combining the antibody of claim 2 with a sample under conditions which allow the formation of antibody:protein complexes; and
b) detecting complex formation, wherein complex formation indicates expression of the protein in the sample.

10. The method of claim 9 wherein complex formation is compared with standards and is diagnostic of a prostatic disorder.

11. The method of claim 9 wherein complex formation is compared with standards and is diagnostic of a gastrointestinal disorder.

12. A composition comprising an antibody of claim 2 and a labeling moiety.

13. A composition comprising an antibody of claim 2 and a pharmaceutical agent.

14. An array containing the antibody of claim 2.

15. A method for treating a prostatic disorder comprising administering to a subject in need of such treatment the antibody of claim 2.

16. A method for treating a gastrointestinal disorder comprising administering to a subject in need of such treatment the antibody of claim 2.

17. A method for testing a molecule or compound for effectiveness as an agonist, the method comprising:
a) exposing a sample comprising a protein of claim 1 to a molecule or compound, and
b) detecting agonist activity in the sample.

18. A purified agonist which modulates the activity of the protein of claim 1.

19. A method for testing a molecule or compound for effectiveness as an antagonist, the method comprising:
a) exposing a sample comprising a protein of claim 1 to a molecule or compound, and
b) detecting antagonist activity in the sample.

20. A purified antagonist which modulates the activity of the protein of claim 1.


SEQUENCE LISTING (8 sequences)

SEQ ID NO: 1 - protein, 283 aa, Homo sapiens
  misc_feature: Incyte ID No: 556016
  unsure 235: unknown or other
MKLNTSAGNVDIYKKLYHSDACSSKAVVSLRCIACGVNLNSSRQSRIVGGESALPGAWPWQVSLHVQNVHVCGGSIITPEWIVTAAHCVEKPLNNPWHWTAFAGILRQSFMFYGAGYQVEKVISHPNYDSKTKNNDIALMKLQKPLTFNDLVKPVCLPNPGMMLQPEQLCWISGWGATEEKGKTSEVLNAAKVLLIETQRCNSRYVYDNLITPAMICAGFLQGNVDSCQGDSGGXLVTSKNNIWWLIGDTSWGSGCAKAYRPGVYGNVMVFTDWIYRQMRADG

SEQ ID NO: 2 - DNA, 1077 bp, Homo sapiens
  misc_feature: Incyte ID No: 556016
  unsure 9-10, 804: a, t, c, g, or other
gcaactctnntacgatcactatagggaaagctggtagcctgcaggtaccggtccggaattcccgggtcgacccacgcgtccgagcggatccaccagctttatgaaactgaacacaagtgccggcaatgtcgatatctataaaaaactgtaccacagtgatgcctgttcttcaaaagcagtggtttctttacgctgtatagcctgcggggtcaacttgaactcaagccgccagagcaggatcgtgggcggcgagagcgcgctcccgggggcctggccctggcaggtcagcctgcacgtccagaacgtccacgtgtgcggaggctccatcatcacccccgagtggatcgtgacagccgcccactgcgtggaaaaacctcttaacaatccatggcattggacggcatttgcggggattttgagacaatctttcatgttctatggagccggataccaagtagaaaaagtgatttctcatccaaattatgactccaagaccaagaacaatgacattgcgctgatgaagctgcagaagcctctgactttcaacgacctagtgaaaccagtgtgtctgcccaacccaggcatgatgctgcagccagaacagctctgctggatttccgggtggggggccaccgaggagaaagggaagacctcagaagtgctgaacgctgccaaggtgcttctcattgagacacagagatgcaacagcagatatgtctatgacaacctgatcacaccagccatgatctgtgccggcttcctgcaggggaacgtcgattcttgccagggtgacagtggagggcntctggtcacttcgaagaacaatatctggtggctgataggggatacaagctggggttctggctgtgccaaagcttacagaccaggagtgtacgggaatgtgatggtattcacggactggatttatcgacaaatgagggcagacggctaatccacatggtcttcgtccttgacgtcgttttacaagaaaacaatggggctggttttgcttccccgtgcatgatttactcttagagatgattcagaggtcacttcatttttattaaacagtgaact

SEQ ID NO: 3 - DNA, 270 bp, Homo sapiens
  misc_feature: Incyte ID No: 556016H1
  unsure 136: a, t, c, g, or other
ccctgaccatctgcttccctgagtacacaggggccaacaaatatgatgaggcagccagctacatccagagtaagtttgaggacctgaataagcgcaaagacaccaaggagatctacacgcacttcacgtgcgccanccgacaaccaagaacgtgcagttcgtgtttgacgccgtcaccgatgtcatcatcaagaacaacctgaaggactgcggcctcttctgaggggcagcggggcctggcgggatgggccaccgccgactttgtacccc

SEQ ID NO: 4 - DNA, 256 bp, Homo sapiens
  misc_feature: Incyte ID No: 842889H1
agaacaatgacattgcgctgatgaagctgcagaagcctctgactttcaacgacctagtgaaaccagtgtgtctgcccaacccaggcatgatgctgcagccagaacagctctgctggatttccgggtggggggccaccgaggagaaagggaagacctcagaagtgctgaacgctgccaaggtgcttctcattgagacacagagatgcaacagcagatatgtctatgacaacctgatcacaccagccatgatctgtgc

SEQ ID NO: 5 - DNA, 294 bp, Homo sapiens
  misc_feature: Incyte ID No: 991163H1
  unsure 8, 65, 73, 90, 172, 179, 192, 199, 223, 241, 259, 263,285: a, t, c, g, or other
ctttacgntgtatagcctgcggggtcaacttgaactcaagccgccagagcaggatcgtgggcggngagagcgngctcccgggggcctggncctggcaggtcagcctgcacgtccagaacgtccacgtgtgcggaggctccatcatcacccccgagtggatcgtgacagccgnccactgngtggaaaaacctnttaacantccatggcattggacggcatttgnggggattttgagacaatntttcatgttctatggagncggntaccaagtagaaaaagtgtttntcatccaaa

SEQ ID NO: 6 - protein, 235 aa, Homo sapiens
  ref <308>: DATABASE ACCESSION NUMBER: Genbank ID No: g416132
IVGGSDSREGAWPWVVALYFDDQQVCGASLVSRDWLVSAAHCVYGRNMEPSKWKAVLGLHMASNLTSPQIETRLIDQIVINPHYNKRRKNNDIAMMHLEMKVNYTDYIQPICLPEENQVFPPGRICSIAGWGALIYQGSTADVLQEADVPLLSNEKCQQQMPEYNITENMVCAGYEAGGVDSCQGDSGGPLMCQENNRWLLAGVTSFGYQCALPNRPGVYARVPRFTEWIQSFLH

SEQ ID NO: 7 - protein, 262 aa, Homo sapiens [sequence omitted]

SEQ ID NO: 8 - protein, 263 aa, Homo sapiens
  ref <308>: DATABASE ACCESSION NUMBER: Genbank ID No: g55527
MWFLILFLALFLGGIDAAPPVQSRIIGGFNCEKNSQPWHVAVYRFARYQCGGVLLDANWVLTAAHCYNDKYQVWLGKNNRFEDEPSAQHQLISKAIPHPGFNMSLLNKDHTPHPEDDYSNDLMLVRLKKPAEITDVVKPIDLPTEEPTVGSRCLASGWGSTTPTEEFEYSHDLQCVYLELLSNEVCAKAHTEKVTDTMLCAGEMDGGKDTCVGDSGGPLICDGVLQGITSWGPTPCALPNVPGIYTKLIEYRSWIKDVMANNP